(19) United States
(12) Patent Application Publication
(10) Pub. No.: US 2026/0257022 A1
(43) Pub. Date: Sep. 3, 2026

(54) MEDICAMENT DELIVERY DEVICE

(71) Applicant: SHL Medical AG, Zug (CH)

(72) Inventors: Oscar Alexandersson, Nacka Strand (SE); Anders Boström, Nacka Strand (SE); Meng-Jhen Chiou, Taoyuan City (TW); Pei Yu Chao, Taoyuan City (TW); Ming-Ting Yin, Taoyuan City (TW); Yi-Ru Cheng, Taoyuan City (TW); Chiao-Yi Chiu, Taoyuan City (TW); Yun Hsuan Chang, Taoyuan City (TW); Chin-Wei Chang, Taoyuan City (TW); Wen-An Chang, Taoyuan City (TW); Tzu-Chia Lee, Taoyuan City (TW); Pei-Hsin Liu, Taoyuan City (TW); Wen-Yen Lee, Taoyuan City (TW); Jung-Chien Chou, Taoyuan City (TW); Wen-Chung Tsai, Nacka Strand (SE); Orshano Kourkis, Nacka Strand (SE)

(21) Appl. No.: 19/163,896

(22) PCT Filed: Mar. 4, 2024
(86) PCT No.: PCT/EP2024/055577

§ 371 Date: Sep. 10, 2025

(30) Foreign Application Priority Data
Mar. 14, 2023 (EP) 23161895.0

(51) Int. Cl. A61M 5/32

(52) U.S. Cl. CPC A61M 5/3202; A61M 2005/3246

(57) ABSTRACT

The present disclosure provides a medicament delivery device including a housing body with a generally tubular shape configured to hold a medicament delivery system and a drive assembly. The medicament delivery device further includes a delivery member cover configured to cover at least a proximal portion of the medicament delivery system and configured to move coaxially within the housing body from a first proximal position to at least a second more distal position and further configured to activate by movement from the first position towards the second position the drive assembly to initiate medicament delivery. A proximal end portion of the delivery member cover comprises an extended diameter portion configured to prevent contact between side surfaces of the device and tissue of an injection site. Alternatively, the medicament delivery device may further comprise a diameter extension part, configured to at least partially surround the delivery member cover and to engage with the proximal end portion of the delivery member cover.

### TECHNICAL FIELD

The present invention relates to a medicament delivery device for obese patients.

### BACKGROUND

Many medicament delivery devices are developed for self-administration, i.e., a user performs the medicament delivery her-, or himself. This requires a medicament delivery device which is as safe to use and as easy to handle as possible.

In order to meet these requirements, the risk of human errors must be minimized, the number of actions needed to be performed in order to receive a dose need to be reduced and the device must be intuitive and ergonomic to use. Thus, in order to minimize the risk of human errors, it is desirable to have the device as pre-assembled as possible.

Medicament delivery devices, such as autoinjectors and manual injectors, often have a needle shield or needle cover that extends in the proximal direction from the housing. The needle shield protects the needle from exposure before medicament delivery. The needle shield may be arranged to move along the longitudinal axis of the housing in a distal direction further into the housing when the medicament delivery device is pushed towards the injection site. The needle is thus exposed so that it can penetrate the patient's skin. For autoinjectors, this movement of the needle shield may cause the medicament delivery device to become activated to discharge medicament via the needle.

US 2014/0114247 A1 relates to an injection device comprising a housing, a container holder arranged within said housing, the container holder being configured for accommodating a medicament container having a needle attached to one end thereof and a stopper sealingly and slidable arranged inside the medicament container at the other end thereof, a drive unit comprising a plunger rod and plunger drive means, the plunger drive means being slidable arranged in relation to the plunger rod, being rotationally locked to the plunger rod and being rotatable in relation to the housing.

US 2019/0022334 A1 relates to a safety device for a pre-filled syringe with an injection needle comprising a support body adapted to mount the pre-filled syringe, a needle shield slidably arranged with respect to the support body, a retention and locking means for retaining and locking the needle shield with respect to the support body in a first and a second advanced position and in a retracted position. The needle shield comprises an annular flange adapted to rest on the skin of the patient receiving an injection.

EP 3007752 A1 relates to an injection device having a contact surface for pressing against the skin of a patient. The contact surface comprises an inwardly extending or radially extending flange or be at the end of a tapered or flared portion of the sliding sleeve to provide an improved contact area.

U.S. Pat. No. 10,398,854 B2 relates to an injector such as a needle-type autoinjector having a flexible flange disposed at an injection end of the injector for stretching or pinching the skin of the injection site. The flexible flange may be removably attached to the injector or integrated into the injector during manufacturing.

### SUMMARY

Many medicament delivery devices are developed for self-administration. If given incorrectly, intramuscular injections may result in poor absorption of drug, reduced drug effectiveness, or irritation to surrounding tissues.

Medicament delivery devices of the types mentioned above, on the market today, do generally not take obese patient skin conditions into account in their designs. For obese patients the amount of subcutaneous tissue present, the weight of the patient, muscle mass, and subcutaneous fat at the injection site may be different. That is, the tissue at the injection site may be softer compared to a normal-weight patient due to the different fat to muscle ratio and thickness of the tissue.

Furthermore, during medicament delivery, the soft skin surrounding the injection site may fold around the at least part of medicament delivery device and a interaction between the medicament delivery device with the folding skin in obese patients may create friction, which results in that a higher activation force is required.

In other words, a larger force is required to push the needle shield into the housing. Some patients with obesity may suffer from impaired dexterity and/or reduced strength, making it challenging to administer medication.

The above problems contribute to a different resistance of the skin against the activation mechanism of the medicament delivery device. Thus, the medicament delivery device may not be activated correctly.

On the other hand, if more pressure is applied to obtain more resistance for activation stretching or bunching of the skin during injection is possible.

With the increasing rates of obesity, caution needs to be taken to determine the amount of subcutaneous fat of the tissue at the injection site, to select the correct needle length, to insert at a predefined angle, and to flatten the skin rather than to bunch the skin.

As mentioned above, a medicament delivery device generally comprises a housing body configured to receive and/or hold the main functional components: an activation system, a medicament delivery system, and a drive assembly.

Furthermore, the housing body may be configured to receive a cap to cover a proximal end portion of the medicament delivery device. The cap is usually removed prior to use of the device.

Many examples of a medicament delivery system are known. Examples are a prefilled syringe based system or a cartridge based system. A medicament delivery system generally comprises a delivery member configured to deliver an injection into the tissue. A typical delivery member is a needle.

In order to deliver an injection, the medicament delivery system generally requires energy. Said energy is provided by the drive assembly. Many examples of a drive assembly are known. Generally, a drive assembly comprises a driver and an energy storage member. In some disclosures the energy storage member is referred to as power pack assembly. In one example the energy storage member is a compression or torsion spring; the driver is moved in a proximal direction and coupled to a plunger of a syringe.

Generally, an activation system is configured to activate the injection, i.e. to affect an energy release from the drive assembly to the medicament delivery system. An example the activation system is a push button at the distal end of the housing body configured to activate the drive assembly.

The present disclosure does not provide a solution for the medicament delivery system, activation system, and/or the drive assembly.

An object of the present disclosure is to provide a medicament delivery device which solves, or at least mitigates, problems of the prior art. The above problems are among those solved by the present disclosure as defined in the appended claims.

In an embodiment of the present disclosure a medicament delivery device that comprises: a housing body with a generally tubular shape configured to hold a medicament delivery system and a drive assembly; and a delivery member cover configured to cover at least a proximal portion of the medicament delivery system and configured to move coaxially within the housing body from a first proximal position to at least a second more distal position and further configured to activate by movement from the first position towards the second position the drive assembly to initiate medicament delivery. A proximal end portion of the delivery member cover comprises an extended diameter portion configured to prevent contact between side surfaces of the device and tissue of an injection site; or the medicament delivery device further comprises a diameter extension part, configured to at least partially surround the delivery member cover and to engage with the proximal end portion of the delivery member cover.

In an embodiment of the present disclosure, the extended diameter portion is formed as an essentially annular flange at the proximal end portion of the delivery member cover.

In an embodiment of the present disclosure, the extended diameter portion comprises cap engagement means configured to engage with corresponding engagement means of a cap in order to hold the cap to the proximal end portion of the delivery member cover.

In an embodiment of the present disclosure, the housing body comprises cap engagement means configured to engage with corresponding engagement means of a cap in order to hold the cap to the proximal end portion of housing body.

In an embodiment of the present disclosure, the extended diameter portion further comprises a sleeve portion extending in a distal direction and configured to at least partially surround the housing body, when the delivery member cover is moved from the first position towards the second position.

In an embodiment of the present disclosure, the sleeve portion comprises cap engagement means configured to engage with corresponding engagement means of a cap in order to hold the cap to the proximal end portion of the housing body; or the sleeve portion is configured to engage via a force-fitting engagement with a cap in order to hold the cap to the proximal end portion of the housing body.

In an embodiment of the present disclosure, the extended diameter portion comprises one or more flexible elements configured to be bent radially outward when a cap is attached to the medicament delivery device.

In an embodiment of the present disclosure, the flexible elements are further configured to lock in a radially fold out position when the cap is removed; or cap holding means are formed on one or more of: the housing body; the delivery member cover; and an inner surface of the cap.

In an embodiment of the present disclosure, the diameter extension part is formed as an essentially circular flange at the proximal end portion of the delivery member cover; or the diameter extension part is formed as an essentially circular flange at the proximal end portion of the delivery member cover having a sleeve portion projection in a distal direction.

In an embodiment of the present disclosure, the diameter extension part comprises a delivery member cover engagement means configured to engage with corresponding engagement means of the delivery member cover in order to prevent the diameter extension part to move further proximally than the proximal end portion of the delivery member cover.

In an embodiment of the present disclosure, the diameter extension part comprises a cap engagement means configured to engage with corresponding engagement means of a cap in order to allow the diameter extension part to follow a movement of the cap.

In an embodiment of the present disclosure, the housing body has a first reduced diameter portion at a proximal end portion thereof; and the extended diameter portion has an inner diameter equal or bigger that the outer diameter of first reduced diameter portion.

In an embodiment of the present disclosure, the housing body has a second reduced diameter portion distally adjacent to the first reduced diameter portion; and the extended diameter portion has an outer diameter essentially the same as the second reduced diameter portion.

In an embodiment of the present disclosure, the outer diameter of the extended diameter portion and the outer diameter of the second reduced diameter portion correspond to an inner diameter of a cap; and/or an outer diameter of the cap is essentially the same as the outer diameter of a remaining portion of the housing body distal to the second reduced diameter portion.

In an embodiment of the present disclosure, the sleeve portion further comprises engagement means for engaging with a rigid needle shield, RNS; and the movement of the delivery member cover is blocked when the engagement means for engaging with a rigid needle shield are engaged.

Based on numerical studies and experimental tests the applicant has realized that the main factor for the optimization of the activation force for obese patients is the ratio between the outer diameter of the housing and the outer diameter of the needle cover, because this factor directly influences the amount of contact between parts of the device and the skin of the injection site.

A further factor is the internal resistance force of the activation mechanism, e.g., the spring force of the needle cover. A lower spring force will reduce the aforementioned problems, however, the optimization is limited because a certain spring force is required to ensure the device functionality as well as to fulfill industry requirements for medicament delivery devices. Thus, in this disclosure this factor is not optimized.

Still a further factor is the offset at full stroke position. The latter being the distance between the contacting surface of the proximal end of the needle cover and the end portion of the housing. It has been found that increasing this distance lowers the activation force. However, at some offset distance, the activation force on soft tissue will plateau due to less resistance because the soft tissue will have less contact with the housing. Thus, in this disclosure this factor is not optimized.

It has further been realized that all three factors may be interrelated, however, to a certain extend they can be independently improved.

It is a first general concept of the disclosure, to provide an extended surface area at the injection site, i.e., the proximal end portion of the needle cover. This is illustrated in FIG. 8.

FIG. 8 illustrates the results of a simulation of an injection with different Models of injectors into obese skin 10. In the columns of FIG. 8 results of four different Models of embodiments of the disclosure are shown. The Models differ in the respective amount of diameter extension. In the rows of FIG. 8 two different amounts of force are applied. All figures show results for the same tissue.

Model #1 has no diameter extension and ratio of 0.79. That is, the outer diameter of the needle cover is smaller than the inner diameter of the housing body.

Model #2 has a diameter extension and a ratio of 0.92. Model 3 has a ratio of 1. Model 4 has a ratio of 1.9.

The simulations show that an increased diameter portion has two main effects.

First, it increases the contact surface with the skin 10 and thus reduces the local pressure on the tissue, which allows for more movement in the activation mechanism of the device at the same pressure. This is evident from a comparison at the solid line P1 of the first row. The needle cover of Model #1 has moved the least and the needle cover of Model #4 has moved the most. However, the needle covers of Model #3 and Model #4 have moved almost the same.

Second, the shape of the folding tissue 10 is changed to an extent that the contact between the skin of the injection site and the moving end portion during activation, i.e., needle shield and/or housing body, is reduced or prevented.

This is evident from the second row of FIG. 8 at the solid line P2. The second row shows a subsequent movement of the housing body 1 during activation. While the needle covers in Model #3 and Model #4 have already reached the respective end position, little to none contact between the tissue 10 and on the side surfaces of the housing body 1 is observed. In Contrast the devices according to Model #1 and Model #2 still require more pressure to reach the end position and exhibit a substantial amount of contact between the tissue 10 at the side surfaces of the housing body. Thus, a ratio of about 1 between the outer diameter of the needle shield 2 and the housing body 1 is a preferred ratio for embodiments of this disclosure. A smaller ratio still allows for some contact with the tissue during activation and a higher ratio does not further optimize the activation force.

The optimum ratio was determined for an essentially flat and circular contact surface with the tissue 10. However, other shapes of contact surfaces are possible. Concave, convex, or complex axial shapes and/or elliptical or complex radial shapes are possible. For said shapes, a comparable ratio is obtained, however, for small deviations from a circular shape the overall effect will be the same.

If the ratio is about 1 and the outer diameter of the housing body is essentially the same as the diameter of the extended surface area portion, the activation force is optimized and the contact with the tissue (except for the intended injection site contact area) is minimized.

It is a second general concept of the disclosure to provide an additional cover, preferably in form of a sleeve, covering a proximal end portion of the needle cover and/or the housing body and thus reduce or prevent the contact with the tissue at the injection site during activation. This is illustrated in FIG. 9.

FIG. 9 shows three different embodiments of the disclosure at the same push pressure. The embodiments on the left, Model #5 and Model #5b are similar to Model #1 and Model #3 as discussed before, respectively. The embodiments of Model #5a has a ratio of about 1. The embodiment Model #6 also has a ratio of about 1, thus it also corresponds to Model #3 as discussed above, but additionally comprises a sleeve portion projecting distally from contact surface. In Model #5a and Model #5b the tissue is in contact with the side surface of the needle cover and the movement of the housing body 1 is not completed, see the two solid lines and the distance A therebetween.

In Model #6, at the same pressure, the skin is only in contact with the sleeve portion and the movement is already completed. Thus, the provision of a sleeve portion further optimizes the activation force.

It is a third general inventive concept of this disclosure, to provide a moving or sliding cover, which may come in contact with the tissue, but does reduce the friction, because the skin is not in contact with the activation mechanism. In this disclosure term “configured to prevent contact” is defined as to be formed such that during the activation movement of the needle cover and the corresponding movement of the housing, in particular the housing body, a contact between the tissue folding around the proximal end surface (see above) and any side surface, i.e. of the needle cover and/or housing body, is essentially prevented or reduced such that the movement is not significantly biased by the friction between the tissue and the contact surface.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings, in which

FIG. 1A shows a side view of a device according to a first embodiment of the present disclosure.

FIG. 1B shows a perspective view of a needle cover of the device according to the first embodiment of the present disclosure.

FIG. 1C shows an enlarged portion of a perspective view of a needle cover of the device according to the first embodiment of the present disclosure.

FIG. 1D shows a perspective view of a cap of the device according to the first embodiment of the present disclosure.

FIG. 1E shows an enlarged portion of a perspective view of the device according to the first embodiment of the present disclosure.

FIG. 2A shows a side view of a device according to a second embodiment of the present disclosure.

FIG. 2B shows a side view of a housing body of the device according to the second embodiment of the present disclosure.

FIG. 2C shows a perspective view of a needle cover of the device according to the second embodiment of the present disclosure.

FIG. 2D shows cross-sectional side view of a cap for the device according to the second embodiment of the present disclosure.

FIG. 2E shows perspective view of a cap for the device according to the second embodiment of the present disclosure.

FIG. 3A shows a side view of a device according to a third embodiment of the present disclosure.

FIG. 3B shows a perspective view of a needle cover of the device according to the third embodiment of the present disclosure.

FIG. 3C shows a cross-sectional perspective view of cap for a device according to the third embodiment of the present disclosure.

FIG. 3D shows a cross-sectional side view of a device according to the third embodiment of the present disclosure.

FIG. 4A shows a perspective view of a device according to a fourth embodiment of the present disclosure.

FIG. 4B shows a perspective view of a needle cover of a device according to the fourth embodiment of the present disclosure.

FIG. 4C shows a cross-sectional perspective view of a cap for a device according to the fourth embodiment of the present disclosure.

FIG. 5A shows a perspective view of a device according to a fifth embodiment of the present disclosure.

FIG. 5B shows an enlarged perspective view of an end portion of a needle cover of a device according to the fifth embodiment of the present disclosure.

FIG. 5C shows a perspective view of a cap for a device according to the fifth embodiment of the present disclosure.

FIG. 6A shows a cross sectional side view of a device according to a sixth embodiment of the present disclosure.

FIG. 6B shows a cross sectional side view of a need cover and a moving element of a device according to a sixth embodiment of the present disclosure.

FIG. 6C shows a perspective view of the needle cover of a device according to a sixth embodiment of the present disclosure.

FIG. 6D shows a perspective view of the moving element of a device according to a sixth embodiment of the present disclosure.

FIG. 6E shows a cross sectional perspective view of an end portion of a device according to a sixth embodiment of the present disclosure with a cap attached.

FIG. 6F shows a cross sectional perspective view of an end portion of a device according to a sixth embodiment of the present disclosure with the cap moved in a proximal direction.

FIG. 6G shows a cross sectional perspective view of an end portion of a device according to a sixth embodiment of the present disclosure with the cap removed.

FIG. 7A shows a cross sectional perspective view of a cap for a device according to a seventh embodiment of the present disclosure.

FIG. 7B shows a cross sectional perspective view of a needle cover of a device according to a seventh embodiment of the present disclosure.

FIG. 7C shows a cross sectional perspective view of a housing body of a device according to a seventh embodiment of the present disclosure.

FIG. 7D shows a cross sectional side view of an end portion of the device with cap according to a seventh embodiment of the present disclosure.

FIG. 8 shows results of a numerical simulation of the activation for different embodiments of the present disclosure.

FIG. 9 shows results of a numerical simulation of the activation for different embodiments of the present disclosure.

FIG. 10A shows a cross sectional side view of an end portion of a device according to an eighth embodiment of the present disclosure.

FIG. 10B shows a perspective view of a rigid needle shield remover of the device according to an eighth embodiment of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

For the purpose of the disclosure the pre-activation is defined as the button is not pressed, and the used pushes the device against their abdomen first. During pre-activation a force is required to bring the device into the first position.

The medicament delivery device of embodiments according to the present disclosure may comprise a housing configured to house the components of a medicament delivery device. The housing may be tubular. The housing may extend along an axis L, which is the longitudinal axis of the tubular shaped housing. The housing has a proximal end and a distal end. The housing has a body which extends between the proximal end and the distal end.

The medicament delivery device may further comprise a delivery member cover, also referred to as needle shield.

The delivery member cover may be generally tubular or tubular. The delivery member cover is arranged in the housing and extends proximally from a proximal opening of the housing body.

The delivery member cover may be configured to be moved linearly relative to the housing body along the axis L. The delivery member cover is configured to be moved linearly from an extended position to a retracted position relative to the housing body. In the retracted position the delivery member cover is received further by the housing.

The medicament delivery device may further comprise a drive assembly (not shown) arranged in the housing. The drive assembly may be any drive assembly used in an autoinjector or a manual injector which activates or triggers medicament delivery by movement of the delivery member cover from the extended position towards the retracted position. Examples of suitable drive assemblies are for example disclosed in WO 2011/123024, WO 2019/011688, WO 2019/011689, WO 2019/011690, and WO 2019/063267 incorporated herein by reference.

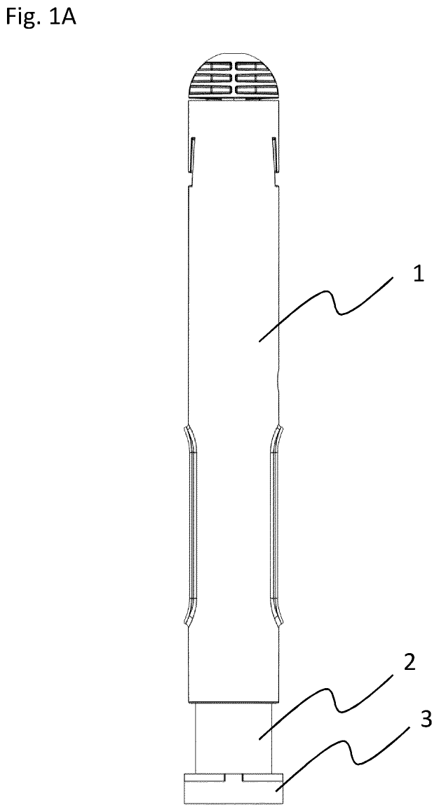

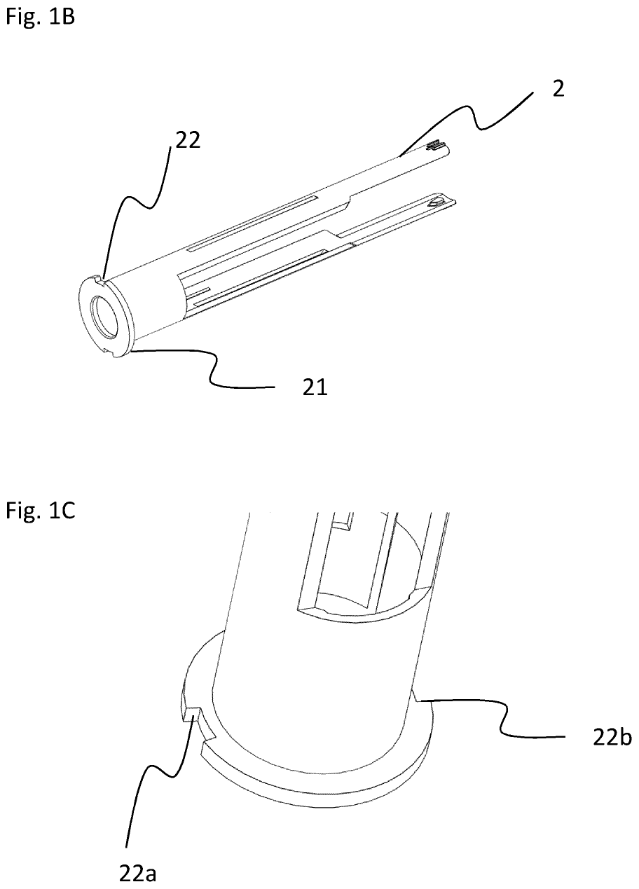

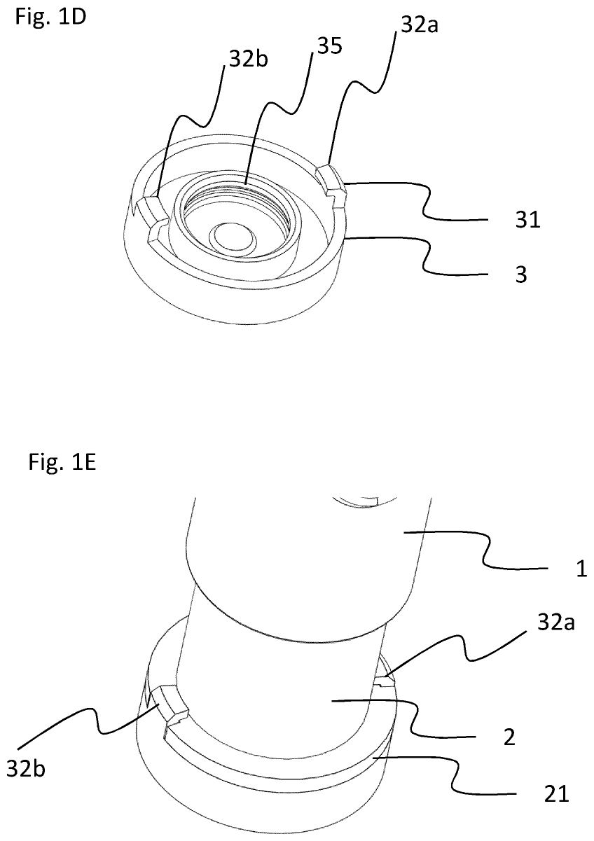

FIGS. 1A to 1E illustrate a first embodiment of the present disclosure. FIG. 1A shows a side view of a device according to a first embodiment of the present disclosure. FIG. 1B shows a perspective view of a needle cover of the device according to the first embodiment of the present disclosure. FIG. 1C shows an enlarged portion of a perspective view of a needle cover of the device according to the first embodiment of the present disclosure. FIG. 1D shows a perspective view of a cap of the device according to the first embodiment of the present disclosure. FIG. 1E shows an enlarged portion of a perspective view of the device according to the first embodiment of the present disclosure.

In the first embodiment of the present disclosure, the device comprises a housing body 1 and a needle shield 2, also referred to as needle cover, which has an enlarged diameter proximal end portion 21. FIG. 1A also shows cap 3. The enlarged diameter proximal end portion 21 is flange shaped. The enlarged diameter proximal end portion 21 is configured to prevent a contact between the tissue at the injection site during the activation movement of the needle cover.

That is, of the outer diameter of the enlarged diameter proximal end portion 21 is essentially equal to the outer diameter of housing body 1, see FIG. 1A. This results in a diameter and/or area ratio of about 1, which is a preferred ratio, as discussed above.

In a preferred embodiment, the enlarged diameter proximal end portion 21 is integrally formed at the needle cover 2. Preferably, the shape of the enlarged diameter proximal end portion 21 is circular and/or annular, respectively.

Variants of this embodiment may have a complex shaped enlarged diameter proximal end portion 21, e.g., elliptical shaped or triangular shape. For said variants the ratio may be defined differently, e.g., based on the enclosed area. Still a ratio of about 1 is preferred, i.e., the area of the contact surface with the tissue is about equal to the area of the proximal end portion of the housing body 1.

In a preferred embodiment, the enlarged diameter proximal end portion 21 of the first embodiment comprises cap engagement means 22, preferably slots 22a and 22b, in the flange portion. A cap 3 comprises corresponding cap engagement means, preferably hooks 32a and 32b (see FIG. 1D), corresponding to the slots 22a and 22b. In a preferred embodiment, the slots and hooks are formed to allow for the cap 3 to snap on the needle cover. This is shown in FIG. 1E.

In a preferred embodiment, at least two slots are formed in the flange. The slots are preferably formed at equal distances along the circumference of the flange 21. The corresponding hooks are formed at corresponding positions on the cap 3. Preferably, there three or four slots and corresponding hooks are formed.

The cap 3 may further comprise further components, e.g., a rigid needle shield, RNS, remover (not shown), mounted to an inner structure 35 of the cap. The further components are configured to prevent movement of the needle cover 2 as long as the cap 3 is attached to the device. That is, accidental activation is prevented when the cap 3 is snapped on.

Alternatively or additionally, a locking mechanism may be provided, which is engaged by the cap 3 and prevents activation when the cap 3 is attached to the device.

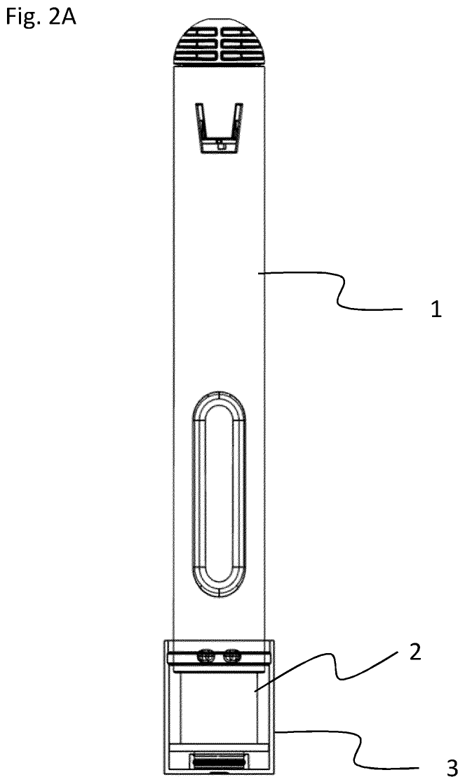

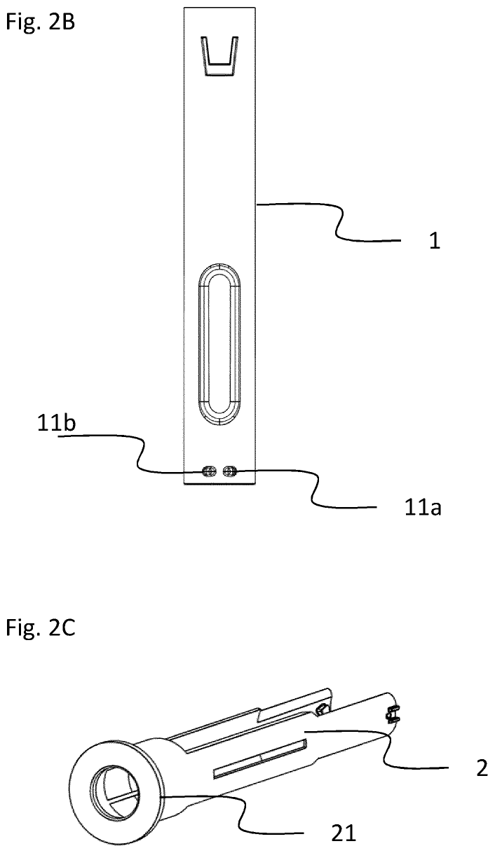

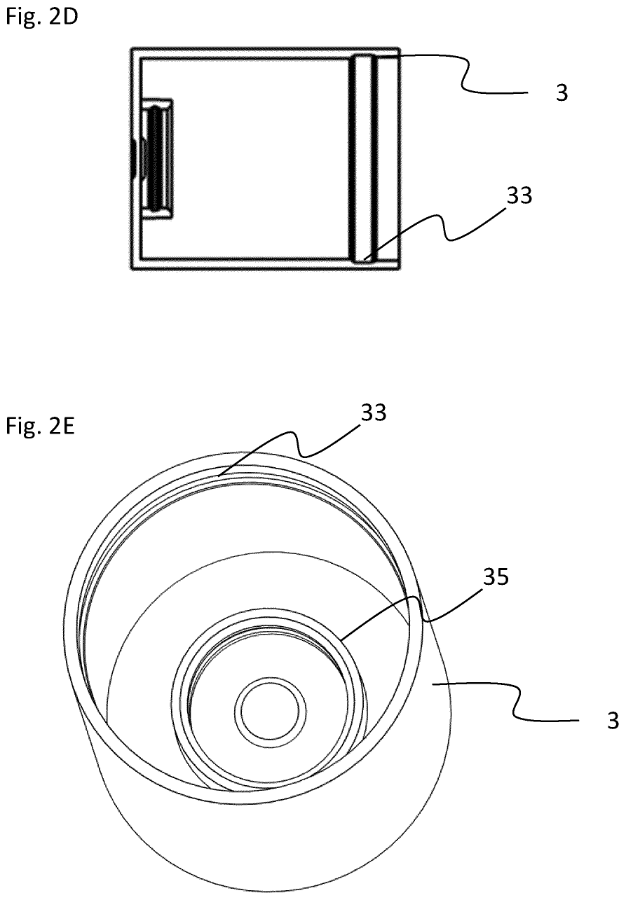

FIGS. 2A to 2E illustrate a second embodiment of the present disclosure. FIG. 2A shows a side view of a device according to a second embodiment of the present disclosure. FIG. 2B shows a side view of a housing body 1 of the device according to the second embodiment of the present disclosure. FIG. 2C shows a perspective view of a needle cover of the device according to the second embodiment of the present disclosure. FIG. 2D shows cross-sectional side view of a cap for the device according to the second embodiment of the present disclosure. FIG. 2E shows perspective view of a cap for the device according to the second embodiment of the present disclosure.

The second embodiment of the present disclosure differs from the first embodiment in that the cap 3 is not snapped to the flange portion 21 but is engaging with the housing body 1. In this embodiment the needle cover 2 is protected by the cap 3. The cap 3 also prevents a movement of the needle cover 2, when the cap 3 is attached to the device. Thus, accidental activation is prevented.

For holding the cap 3 to the housing body 1, the housing body 1 and the cap 3 comprise corresponding engagement means. In a preferred embodiment, the housing body 1 comprises knobs 11a and 11b (see FIG. 2B) and the cap 3 comprises at least one corresponding recess 33 at an inner surface of the cap 3 (see FIG. 2E).

In a preferred embodiment, the cap 3 comprises at least one corresponding recess 33 formed as a groove at the inner surface of the cap 3. This way, the cap 3 can engage with the knobs 11a, 11b in any rotational orientation.

In a preferred embodiment, two pairs of knobs 11a, 11b are provided.

Alternatively or additionally, a locking mechanism may be provided, which is engaged by the cap 3 and prevents activation when the cap 3 is attached to the device.

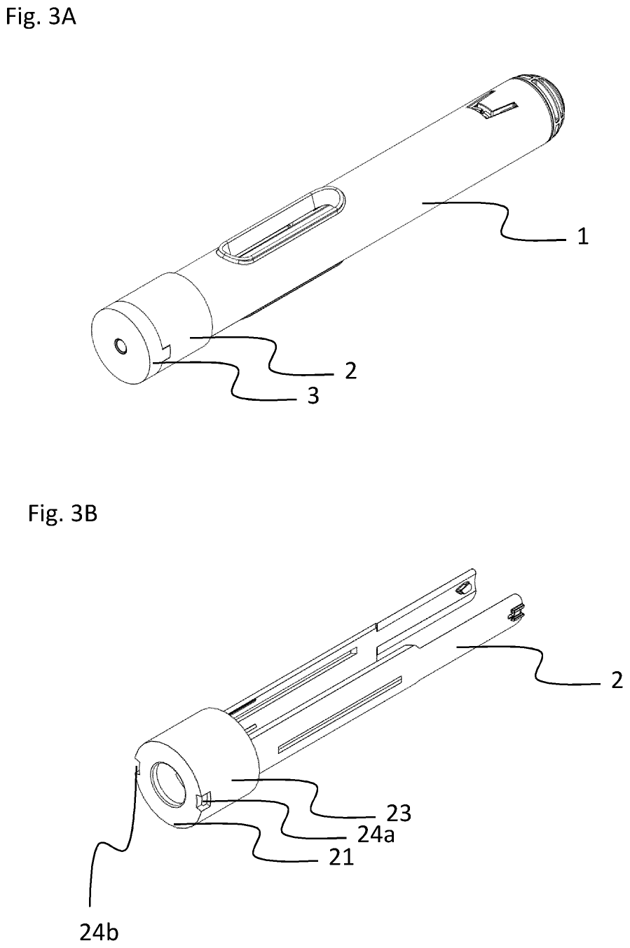

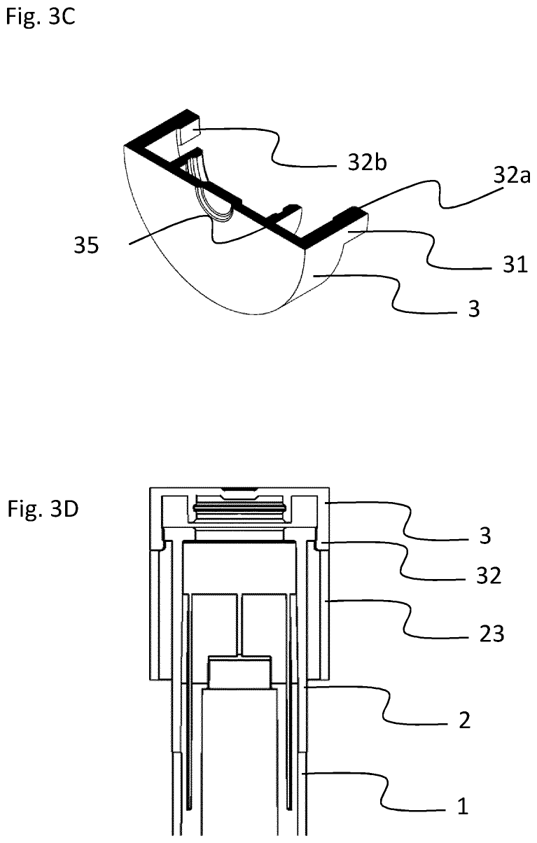

FIGS. 3A to 3D illustrate a third embodiment of the present disclosure. FIG. 3A shows a side view of a device according to a third embodiment of the present disclosure. FIG. 3B shows a perspective view of a needle cover of the device according to the third embodiment of the present disclosure. FIG. 3C shows a cross-sectional perspective view of cap for a device according to the third embodiment of the present disclosure. FIG. 3D shows a cross-sectional side view of a device according to the third embodiment of the present disclosure.

The third embodiment is based on the first embodiment as described above. In the third embodiment, an enlarged diameter proximal end portion 21 is provided with a sleeve portion 23 projecting into a distal direction. The sleeve portion 23 is configured to, at least partially, enclose the housing body 1 during the movement of the needle shield 2.

That is, the inner diameter of the sleeve portion 23 is bigger that the inner diameter of the housing body 1. At the same time the outer diameter of the sleeve portion 23 still is essentially equal to the outer diameter of the housing body 1. In other words, it is preferred that a wall thickness of the sleeve portion 23 is small compared to the outer diameter of the housing body 1 and the sleeve portion 23.

In a preferred embodiment, the length in an axial direction of the sleeve portion 23 is defined by the maximal movement distance of the needle cover 2, since the sleeve portion is configured to, at least partially, surround the proximal end portion of the housing body 1 during the movement.

As detailed above, the sleeve portion 23 may come in contact with the tissue of the injection site but prevents a contact of the housing body 1 with the tissue and thus reduces the activation force.

In an alternative embodiment, the sleeve portion 23 does not surround the housing body 1 during a portion or during the entire movement thereof. However, in this embodiment the sleeve portion 23 is formed sufficiently long in order to prevent contact between the housing body 1 and the tissue during the movement.

In a preferred embodiment, the needle cover 2 comprises cap engagement means configured to engage with corresponding engagement means at a cap 3. Preferably two or more slots 24a and 24b (see FIG. 3B) are formed as cap engagement means in a proximal end portion of the sleeve portion 23. Accordingly, two or more hooks 32a and 32b are formed on protrusions 31 on the cap 3 (FIG. 3C).

As detailed above, the cap 3 further comprises an inner structure 35 configured to hold further components, such as a RNS remover. The further components prevent a movement of the needle cover 2, when the cap 3 is attached to the device.

Alternatively or additionally, a locking mechanism may be provided, which is engaged by the cap 3 and prevents activation when the cap 3 is attached to the device.

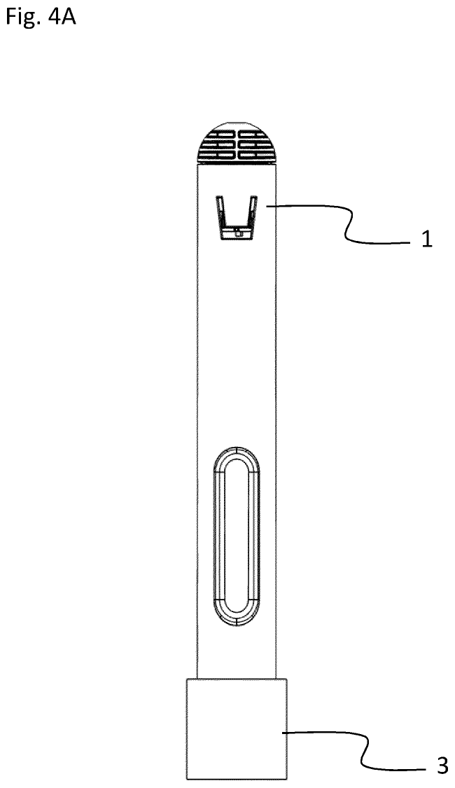

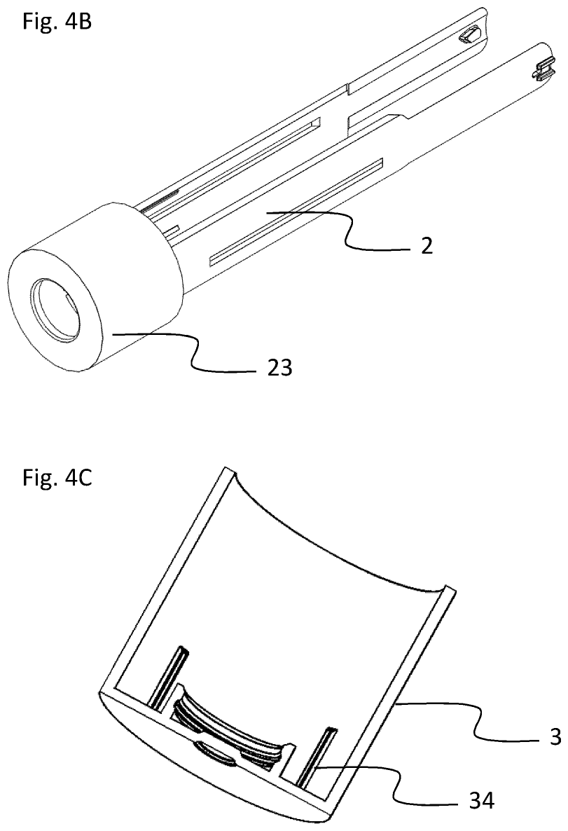

FIGS. 4A to 4C illustrate a fourth embodiment of the present disclosure. FIG. 4A shows a side view of a device according to a fourth embodiment of the present disclosure. FIG. 4B shows a side view of a needle cover of a device according to the fourth embodiment of the present disclosure. FIG. 4C shows a cross-sectional side view of a cap for a device according to the fourth embodiment of the present disclosure.

The fourth embodiment is based on the third embodiment. The difference is that the sleeve portion 23 of the fourth embodiment does not comprise cap engagement means.

In the fourth embodiment the cap 3 and the sleeve portion 23 engage via cap engagement means formed on the inner surface of the cap 3.

In a preferred embodiment, the cap 3 and the sleeve portion 23 engage via a clamp and/or press connection, i.e., via force fitting.

In a preferred embodiment, at least two protrusions 34 are provided on an inner surface of the cap 3. The protrusions extend in axial direction. The protrusions 34 are configured to force-fit to the sleeve portion 23 and thus engage the cap 3 and the sleeve portion 23. The protrusions 34 may be spaced from each other in circumferential direction at equal distances. Preferably, the protrusions are formed at a proximal portion of the cap 3.

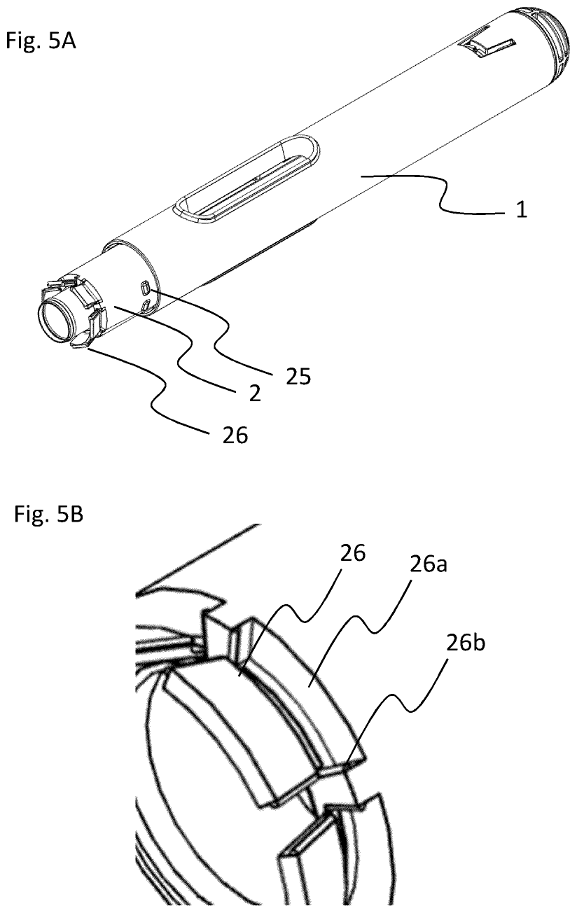

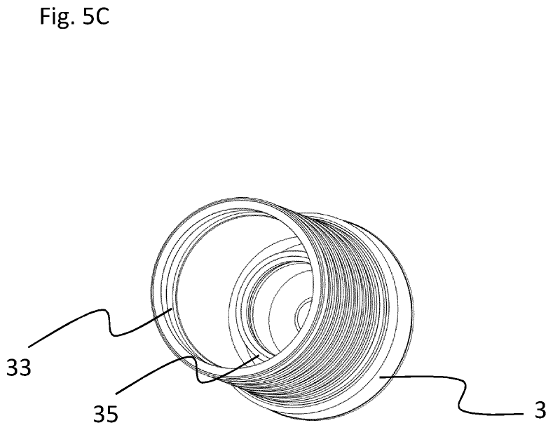

FIGS. 5A to 5C illustrate a fifth embodiment of the present disclosure. FIG. 5A shows a perspective view of a device according to a fifth embodiment of the present disclosure. FIG. 5B shows an enlarged perspective view of an end portion of a needle cover of a device according to the fifth embodiment of the present disclosure. FIG. 5C shows a perspective view of a cap for a device according to the fifth embodiment of the present disclosure.

The needle cover 2 of the fifth embodiment comprises cap engagement means 25 formed at a circumferential sidewall thereof and further comprises at least two flexible arms 26, forming the enlarged diameter proximal end portion when folded outward. The flexible arms 26 protrude in proximal direction.

Preferably, the flexible arms are six arms, more preferably more than six arms, and the arms are essentially equidistantly spaced along the circumference of the end portion of the needle cover 2.

The flexible arms 26 are configured to fold radially outward, when the proximal end portion of the needle cover 2 is brought in contact with the tissue at the injection surface. When the flexible arms 26 are in the fold-out position the arms 26 form an enlarged diameter proximal end portion of the needle cover 2 corresponding to the flange portion of the first embodiment.

In particular, the outer diameter of the flange formed by the fold-out flexible arms 26 is configured to prevent contact of the housing body with the tissue of the injection site during activation. That is, the outer diameter is essentially the same as the outer diameter of the proximal end portion of the housing body 1.

The flexible arms 26 are further configured to fold inwardly, i.e., towards the axial center of the device, when the device is not in contact with the tissue of the injection site, e.g., during storage.

In a preferred embodiment, the flexible arms 26 are integrally formed with the needle cover 2. In this embodiment the flexible arms 26 are in the fold-in position due to the material stiffness of the needle cover 2 and they fold out due to the material flexibility of a connection or linking portion between the flexible arms 26 and the needle cover 2.

In a preferred embodiment, the movement of the flexible arms 26 is limited by a contacting or abutment surface 26a at the end portion of the needle cover 2. Additionally or alternatively, the flexible arms 26 and/or the end portion of the needle cover 2 are configured to engage via corresponding engagement means 26b to lock the flexible arms 26 in the fold-out position. This improves the handling of the device and indicates that the device is ready to use.

In the fifth embodiment, when the flexible arms 26 are in the fold-in position, the diameter of the end portion of the needle cover 2 is not increased. Therefore, a normal size cap may be used.

The cap 3 may engage with the needle cover, see FIGS. 5A and 5C or may engage with the housing body 1, equivalent to the second embodiment. In a preferred embodiment, the aforementioned engagement is based on knobs 25 being formed at the side surface of the needle cover 2 and/or the housing body 1; and a corresponding recess structure, preferably a groove 33 being formed in the cap 3. This way, the cap 3 may be attached in any orientation.

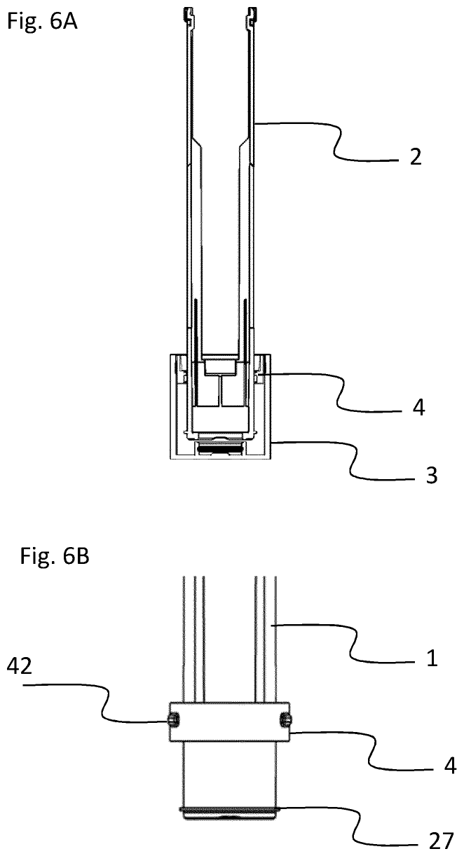

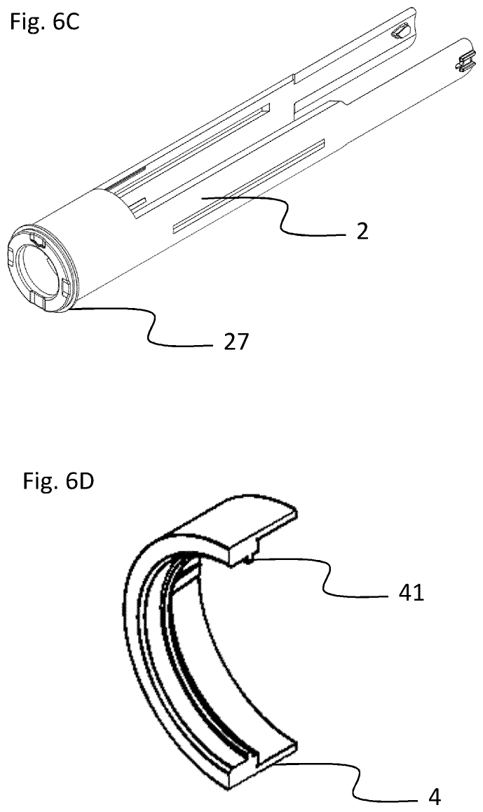

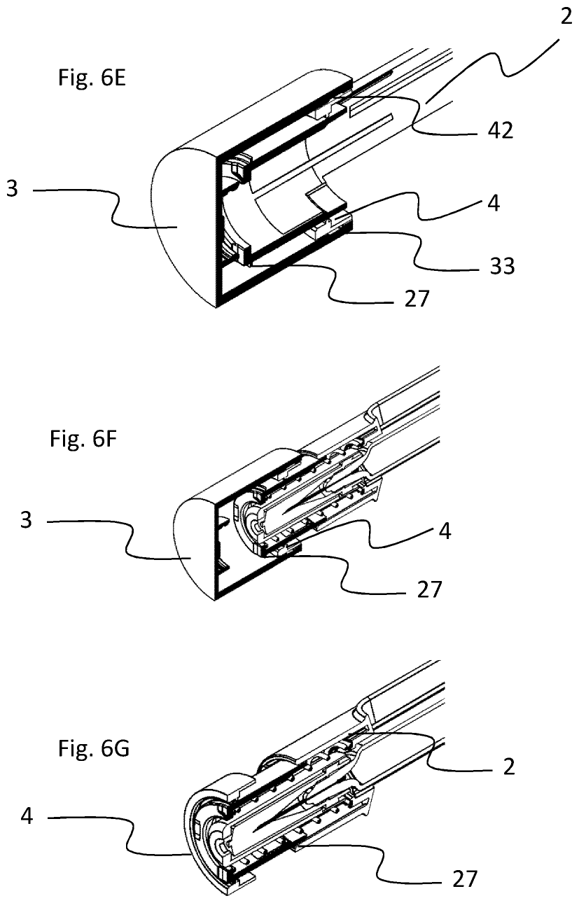

FIGS. 6A to 6D illustrate a sixth embodiment of the present disclosure. FIG. 6A shows a cross sectional side view of a device according to a sixth embodiment of the present disclosure. FIG. 6B shows a cross sectional side view of a need cover and a moving element of a device according to a sixth embodiment of the present disclosure. FIG. 6C shows a perspective view of the needle cover of a device according to a sixth embodiment of the present disclosure. FIG. 6D shows a perspective view of the moving element of a device according to a sixth embodiment of the present disclosure.

FIGS. 6E to 6F illustrate the cap removal according to the sixth embodiment of the present disclosure. FIG. 6E shows a cross sectional perspective view of an end portion of a device according to a sixth embodiment of the present disclosure with a cap attached. FIG. 6F shows a cross sectional perspective view of an end portion of a device according to a sixth embodiment of the present disclosure with the cap moved in a proximal direction. FIG. 6G shows a cross sectional perspective view of an end portion of a device according to a sixth embodiment of the present disclosure with the cap removed.

In the sixth embodiment the enlarged diameter end portion is not integrally formed with the proximal end of the needle cover 2. Instead, the enlarged diameter end portion is provided as a moving element 4 configured to move on the needle cover 2 and configured to lock on the proximal end of the needle cover 2 at the during the removal of a cap, see FIGS. 6E to 6F.

In a preferred embodiment, the moving element 4 is shaped equivalent to the flange in the first and second embodiment or as a flange with sleeve portion equivalent to the flange and sleeve in the third and fourth embodiment.

In the sixth embodiment the inner diameter of the moving element 4 corresponds to the outer diameter of the needle cover 2, such that the moving element 4 can move or slide on the side surface of the needle cover 2.

In the sixth embodiment the proximal end portion of the moving element 4 is configured to engage with the proximal end portion of the needle cover 2. Preferably, a corresponding engagement means 41 are formed on the moving element 4 and the needle cover flange 27. The engagement prevents the moving element 4 from sliding off the end portion of the needle cover 2 and optionally locks the moving element 4 to the end portion of the needle cover 2.

Preferably an inner flange 41 is formed on the inner surface of the moving element 4 and/or an outer flange 27 is formed on the outer surface of the needle cover 2.

In a preferred embodiment, the moving element 4 further comprises engagement means 42 configured to engage with corresponding engagement means of a cap 3. Preferable the engagement means 42 are two or more knob or sets of knobs formed on a side surface of the moving element. The knobs 42 preferably engage with a corresponding structure on the cap 3, preferably a recess as discussed above and shown, e.g., in FIG. 5A.

In the sixth embodiment, when the cap 3 is attached to the device, the cap 3 and the moving element 4 are engaged and the moving element 4 is located in a distal position at the position of the engagement means of the cap 3, preferably at a distal end portion of the cap, this is shown in FIG. 6E.

In order to remove the cap 3, a user may pull the cap 3 into a proximal direction. The cap 3 and the moving element 4 are still engaged and the moving element 4 slides in a proximal direction with the cap 3, this is shown in FIG. 6F.

When the moving element 4 reaches the proximal end of the needle cover 2, the engagement means 27 and 41 engage and the moving element 4 cannot move any further in a proximal direction, preferably the moving element 4 locks to the needle cover 2 in this position.

As the user continuous to pull the cap 3 in a proximal direction, the engagement between the cap 3 and the moving element 4 is released, and the cap 3 detaches from the device. The moving element 4 remains at the proximal end portion of the needle cover 2 and forms the enlarged diameter end portion as discussed in the above. This is shown in FIG. 6G.

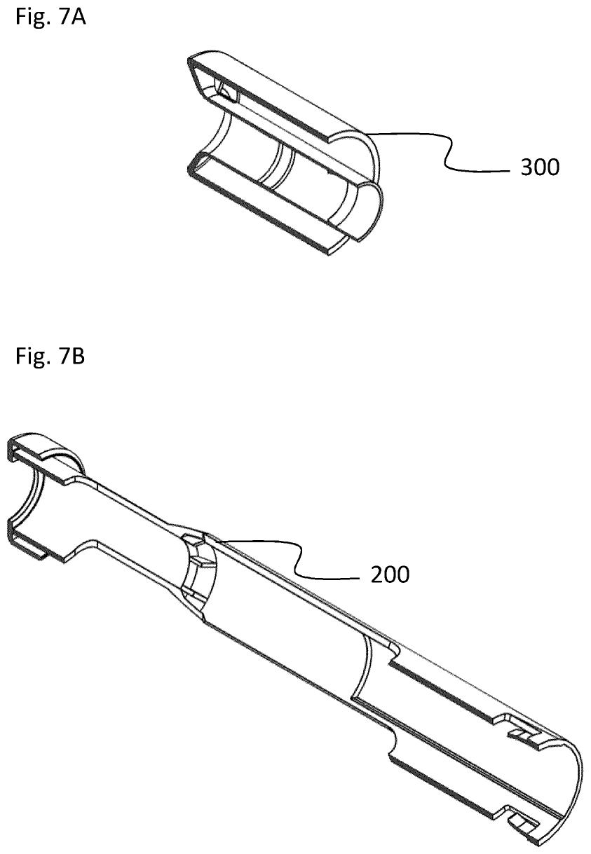

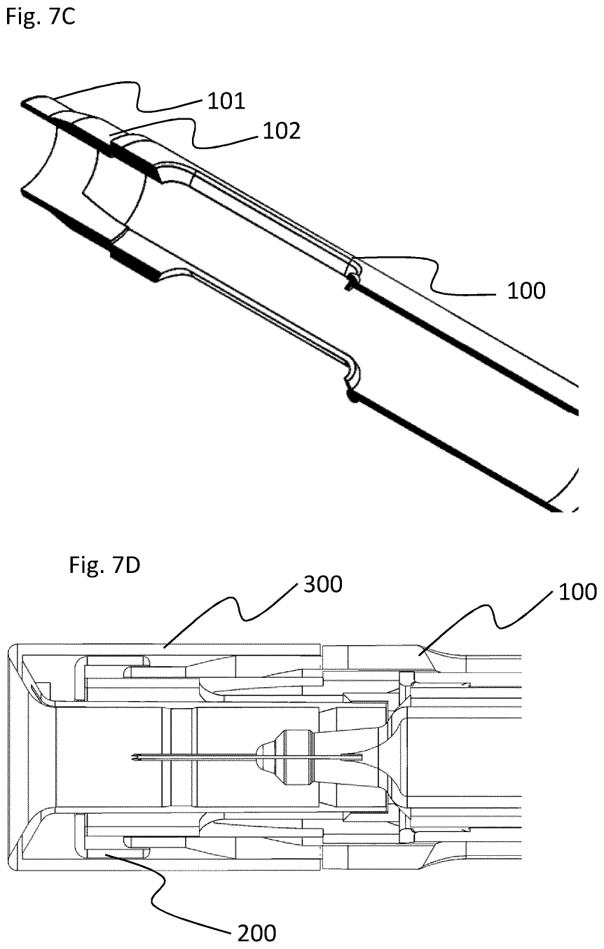

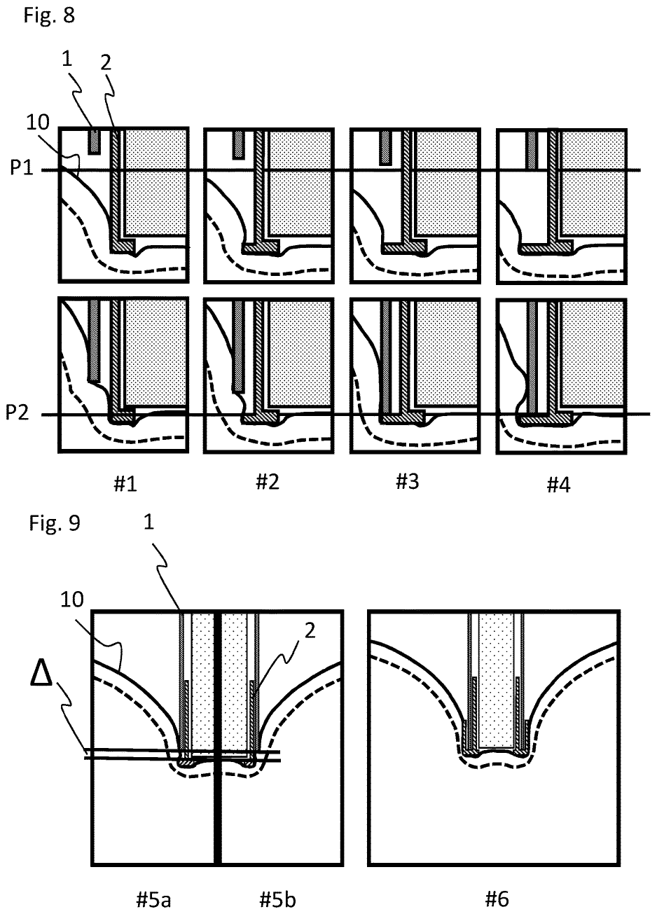

FIGS. 7A to 7D illustrate a seventh embodiment of the present disclosure. FIG. 7A shows a cross sectional perspective view of a cap for a device according to a seventh embodiment of the present disclosure. FIG. 7B shows a cross sectional perspective view of a needle cover of a device according to a seventh embodiment of the present disclosure. FIG. 7C shows a cross sectional perspective view of a housing body of a device according to a seventh embodiment of the present disclosure. FIG. 7D shows a cross sectional side view of an end portion of the device with cap according to a seventh embodiment of the present disclosure.

The seventh embodiment is based on the third and fourth embodiment. In the seventh embodiment the housing body 100 has a first reduced outer diameter portion 101 at the proximal end, the needle cover 200 has a flange and sleeve portion with an outer diameter corresponding to the outer diameter of the housing body 100 at the end portion, such that the sleeve portion fits over the end portion of the housing body 100.

Furthermore, the housing body 100 comprises a second reduced diameter portion 102 distal to the first reduced diameter portion 101. preferably the is a gradual increase of the outer diameter between the first and second reduced diameter portion.

The outer diameter of the sleeve portion and the outer diameter of the second reduced diameter portion are essentially the same. This provided for an optimized activation force as discussed above. Furthermore, the sleeve and the second reduced diameter portion provide an essentially flat surface for a cap 300.

The cap 300 is formed to have an inner diameter corresponding to the outer diameter of the sleeve portion and the outer diameter of the second reduced diameter portion 102. Preferably, the cap 300 has an outer diameter corresponding to the outer diameter of the remaining housing.

In a preferred embodiment, the needle cover 200 and or the housing body 100 comprises cap engagement means as discussed for any of the above embodiments.

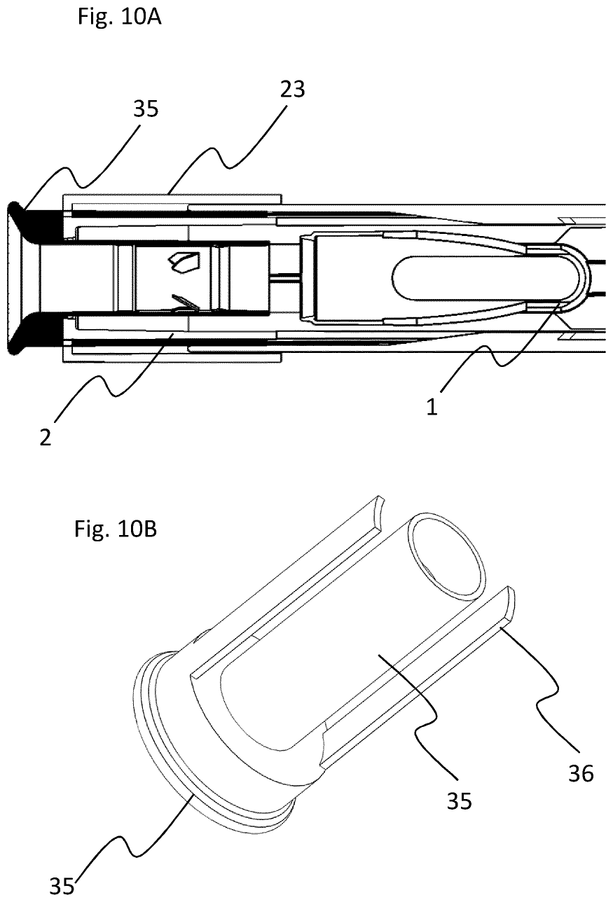

FIGS. 10A and 10B illustrate an eighth embodiment of the present disclosure. FIG. 10 shows a cross sectional side view of an end portion of a device according to an eighth embodiment of the present disclosure. FIG. 10B shows a perspective view of a rigid needle shield remover of the device according to an eighth embodiment of the present disclosure.

The eighth embodiment is based on the third and fourth embodiment. The core concept of the eighth embodiment is to integrate the cap functionality into the needle cover. In other words, the sleeve portion 23 is further configured to prevent a contact between the folding tissue and the housing body 1.

Furthermore, the sleeve portion 23 is provided with at least two slots for receiving a corresponding number of protrusions 36 of a rigid needle shield, RNS, remover 35.

In other words, only the RNS remover 35 is acting as cap to be removed before using the device.

It has been shown that this design eliminates encapsulation of soft tissue and/or skin between needle cover 2 and/or the housing body 1. The test results in the lab have shown that device according to the eighth design has the same activation force on soft tissue as it has on a steel plate.

In a preferred embodiment the cap integrated needle cover is configured to be locked in the axial direction when the protrusions 36 are inserted into the sleeve portion 23. The movement of the needle shield 2 is only possible when the RNS remover 36 has been removed.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjogren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Various modifications to the embodiments described are possible and will occur to those skilled in the art without departing from the invention which is defined by the following claims.

1-15. (canceled)

16. A medicament delivery device comprising:
a housing body with a generally tubular shape configured to hold a medicament delivery system and a drive assembly; and
a delivery member cover configured to cover at least a proximal portion of the medicament delivery system and configured to move coaxially within the housing body from a first proximal position to at least a second more distal position and further configured to activate by movement from the first position towards the second position the drive assembly to initiate medicament delivery,
wherein a proximal end portion of the delivery member cover at comprises an extended diameter portion configured to prevent contact between side surfaces of the device and tissue of an injection site, or
wherein the medicament delivery device further comprises a diameter extension part, configured to at least partially surround the delivery member cover and to engage with the proximal end portion of the delivery member cover.

17. The medicament delivery device according to claim 16,
wherein the extended diameter portion is formed as an essentially annular flange at the proximal end portion of the delivery member cover.

18. The medicament delivery device according to claim 17,
wherein the extended diameter portion comprises cap engagement means configured to engage with corresponding engagement means of a cap in order to hold the cap to the proximal end portion of the delivery member cover.

19. The medicament delivery device according to claim 17,
wherein the housing body comprises cap engagement means configured to engage with corresponding engagement means of a cap in order to hold the cap to the proximal end portion of housing body.

20. The medicament delivery device according to claim 16,
wherein the extended diameter portion further comprises a sleeve portion extending in a distal direction and configured to at least partially surround the housing body, when the delivery member cover is moved from the first position towards the second position.

21. The medicament delivery device according to claim 20,
wherein the sleeve portion comprises cap engagement means configured to engage with corresponding engagement means of a cap in order to hold the cap to the proximal end portion of the housing body, or

[symbol]

22. The medicament delivery device according to claim 20,
wherein the sleeve portion is configured to engage via a force-fitting engagement with a cap in order to hold the cap to the proximal end portion of the housing body.

23. The medicament delivery device according to claim 16,
wherein the extended diameter portion comprises one or more flexible elements configured to be bent radially outward when a cap is attached to the medicament delivery device.

24. The medicament delivery device according to claim 23,
wherein the flexible elements are further configured to lock in a radially fold out position when the cap is removed.

25. The medicament delivery device according to claim 23,
wherein cap engagement means are formed on one or more of:
the housing body;
the delivery member cover; and
an inner surface of the cap.

26. The medicament delivery device according to claim 16,
wherein the diameter extension part is formed as an essentially circular flange at the proximal end portion of the delivery member cover.

27. The medicament delivery device according to claim 16,
wherein the diameter extension part is formed as an essentially circular flange at the proximal end portion of the delivery member cover having a sleeve portion projection in a distal direction.

28. The medicament delivery device according to claim 26,
wherein the diameter extension part comprises a delivery member cover engagement means configured to engage with corresponding engagement means of the delivery member cover in order to prevent the diameter extension part to move further proximally than the proximal end portion of the delivery member cover.

29. The medicament delivery device according to claim 26,
wherein the diameter extension part comprises a cap engagement means configured to engage with corresponding engagement means of a cap in order to allow the diameter extension part to follow a movement of the cap.

30. The medicament delivery device according to claim 16,
wherein the housing body has a first reduced diameter portion at a proximal end portion thereof, and
wherein the extended diameter portion has an inner diameter equal or bigger that the outer diameter of first reduced diameter portion.

31. The medicament delivery device according to claim 30,
wherein the housing body has a second reduced diameter portion distally adjacent to the first reduced diameter portion, and
wherein the extended diameter portion has an outer diameter essentially the same as the second reduced diameter portion.

32. The medicament delivery device according to claim 31,
wherein the outer diameter of the extended diameter portion and the outer diameter of the second reduced diameter portion correspond to an inner diameter of a cap, and/or
wherein an outer diameter of the cap is essentially the same as the outer diameter of a remaining portion of the housing body distal to the second reduced diameter portion.

33. The medicament delivery device according to claim 20,
wherein the sleeve portion further comprises engagement means for engaging with a rigid needle shield, and
wherein the movement of the delivery member cover is blocked when the engagement means for engaging with a rigid needle shield are engaged.
